(19) United States
(12) Patent Application Publication
(10) Pub. No.: US 2011/0054510 A1
(43) Pub. Date: Mar. 3, 2011

(54) SYSTEM AND METHOD FOR ASSEMBLING A LANCING DEVICE USING A SPRING WINDING FIXTURE

(75) Inventors: Lauren R. PUSEY, Woodstock, GA (US); Brian M. COLLINS, Denver, CO (US)

(73) Assignee: FACET TECHNOLOGIES, LLC, Kennesaw, GA (US)

(21) Appl. No.: 12/872,887

(22) Filed: Aug. 31, 2010

Related U.S. Application Data
(60) Provisional application No. 61/239,247, filed on Sep. 2, 2009.

(51) Int. Cl. A61B 5/151; B23P 19/04

(52) U.S. Cl. 606/182; 29/240.5; 29/428

(57) ABSTRACT

A system and method for assembling a lancing device, and a lancing device fabricated according to such a system or method. A rotating disk is applied to a spring component and/or to a carrier component of the lancing device to mount the spring onto the carrier component.

### CROSS-REFERENCE TO RELATED APPLICATION

This application claims the benefit of U.S. Provisional Patent Application Ser. No. 61/239,247, filed Sep. 2, 2009, the entirety of which is hereby incorporated herein by reference for all purposes.

### TECHNICAL FIELD

The present invention relates generally to the field of manufacturing and medical devices; and more particularly to systems and methods of manufacturing lancing devices, and to lancing devices produced thereby.

### BACKGROUND

Lancing devices used for blood sampling commonly include one or more springs for driving and/or returning the lancet or lancet carrier along its lancing stroke. Assembly of such lancing devices often requires manipulation of the spring(s) for attachment to associated components. It is to the provision of improved systems and methods of assembling lancing devices, and to lancing devices assembled using such systems and methods that the present invention is primarily directed.

### SUMMARY

In example embodiments, the present invention provides improved systems and methods of assembling lancing devices that include springs mounted or connected to associated component parts. The invention also includes lancing devices assembled using such systems and methods. Example forms of the invention enable production of such lancing devices more efficiently and cost effectively than previously known methods, including entirely manual methods of assembly, and/or provide a more consistent assembly for improved product quality

In one aspect, the present invention relates to a system for assembly of a lancing device. The lancing device includes a carrier component and at least one spring to be mounted to the carrier component. The system includes a disk for engaging at least a portion of the spring, and a drive mechanism for rotationally driving the disk, wherein the disk has a hardness selected to grip the spring and mount it onto the lancet carrier.

In another aspect, the present invention relates to a system for assembly of a device having a carrier component comprising a shaft and a retainer portion at an end of the shaft, and further comprising a spring mounted onto the shaft of the carrier component. The system preferably includes a disk having an outer radial contact face comprising a material having a Shore A durometer of between about 35-95, and a motor coupled to the disk to rotationally drive the disk. Application of the spring against the contact face of the disk spins the spring over the retainer portion of the carrier component and onto its shaft.

In another aspect, the present invention relates to a method of assembly of a lancing device. The assembly method includes applying a spring into partial assembly with a carrier component of a lancing device, engaging the spring with a rotating disk portion of a lancing device assembly system, and further assembling the spring onto the carrier component by operation of the rotating disk portion on the spring.

In still another aspect, the invention relates to a lancing device including a carrier component and at least one spring, wherein the lancing device is assembled by installing the at least one spring onto the carrier component by winding the spring onto the carrier component using a rotating disk that is applied against at least a portion of the spring and/or against at least a portion of the carrier component.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a lancing device component having a spring partially mounted thereon, assembled according to an example embodiment of the present invention.

FIG. 2 shows the assembly of FIG. 1, with the spring completely mounted to the lancing device component.

FIG. 3 shows a spring winding device according to an example embodiment of the present invention.

FIG. 4 is an assembly view of the spring winding device of FIG. 3.

FIG. 5 shows installation of a spring onto a lancet carrier portion of a lancing device using an example form of the assembly system and method of the present invention.

FIG. 6 shows the spring installed onto the lancet carrier.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

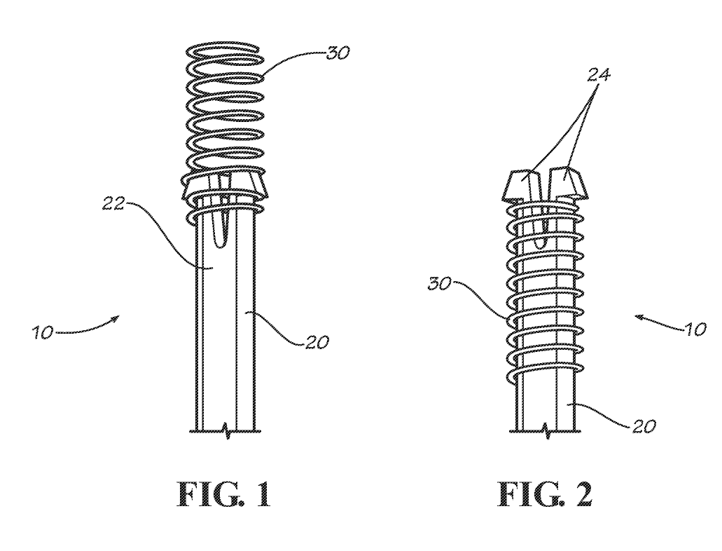

With reference now to the drawing figures, wherein like reference numbers represent corresponding parts throughout the several views, FIGS. 1 and 2 show an example form of a lancing device subassembly 10 which may be fabricated using the assembly system and method of the present invention. The lancing device subassembly 10 includes a lancet carrier, a spring carrier, or other lancing device component 20 and one or more springs 30 for actuating the lancet carrier within an assembled lancing device to advance and retract a lancet carried by the lancet carrier along its lancing stroke in typical fashion, as for blood typing, sampling, or other medical procedure(s).

The lancet carrier component 20 generally comprises an elongate shaft 22 having a medial portion onto which the spring 30 is mounted, and an expanded end portion comprising one or more barbs or retainers 24. The end portion optionally comprises a split-yoke configuration having a pair of resilient forks with a slot extending therebetween, to allow the forks to compress inwardly during installation of the spring 30 and return under the bias of their resilient material of construction to an expanded equilibrium position. The barbs or retainers 24 at the ends of the forks optionally comprise opposed one-way ramped or inclined outwardly directed distal surfaces that compress the forks inwardly as the spring 30 is installed, and flat or acutely angled proximal shoulders for retaining the spring on the shaft after assembly. In the depicted embodiment, the lancet carrier 20 also includes a cylindrical receiver or resilient collar 25 opposite the forked end for releasably engaging a disposable lancet, a cantilevered arm 26 extending from a medial portion of the shaft 22 between the cylindrical receiver and the forked end, a circumferential flange 27 projecting outwardly from a medial shaft portion between the cylindrical receiver and the forked end, and a spring retainer tab 28 projecting from the shaft adjacent the flange. While the depicted embodiment shows a device subassembly comprising a lancet carrier component of a lancing device, it will be understood that the system and method of the invention may be adapted for use in assembly of various other types of components, subassemblies and/or devices wherein a spring is mounted onto, into, or otherwise operatively coupled to or with a component.

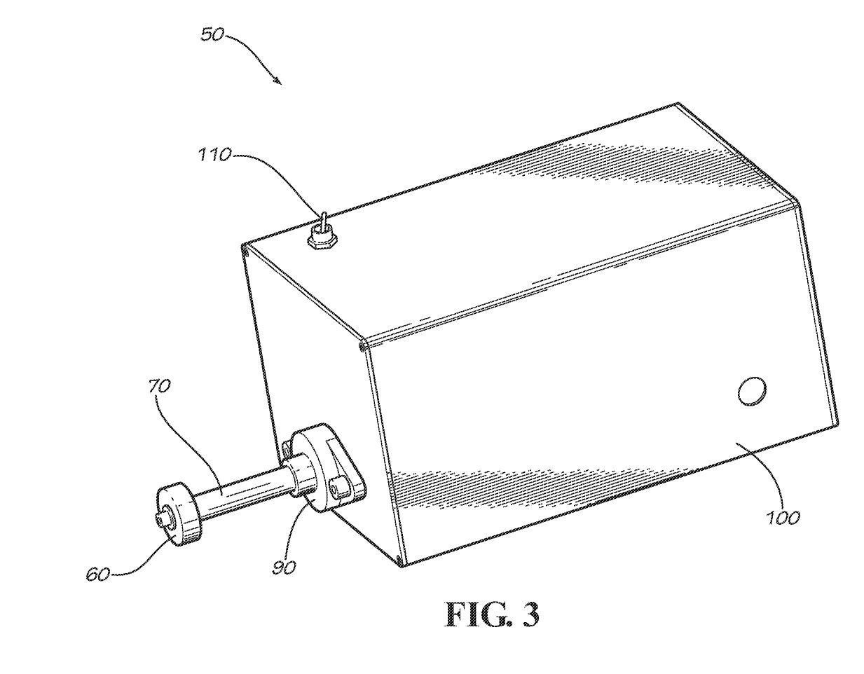

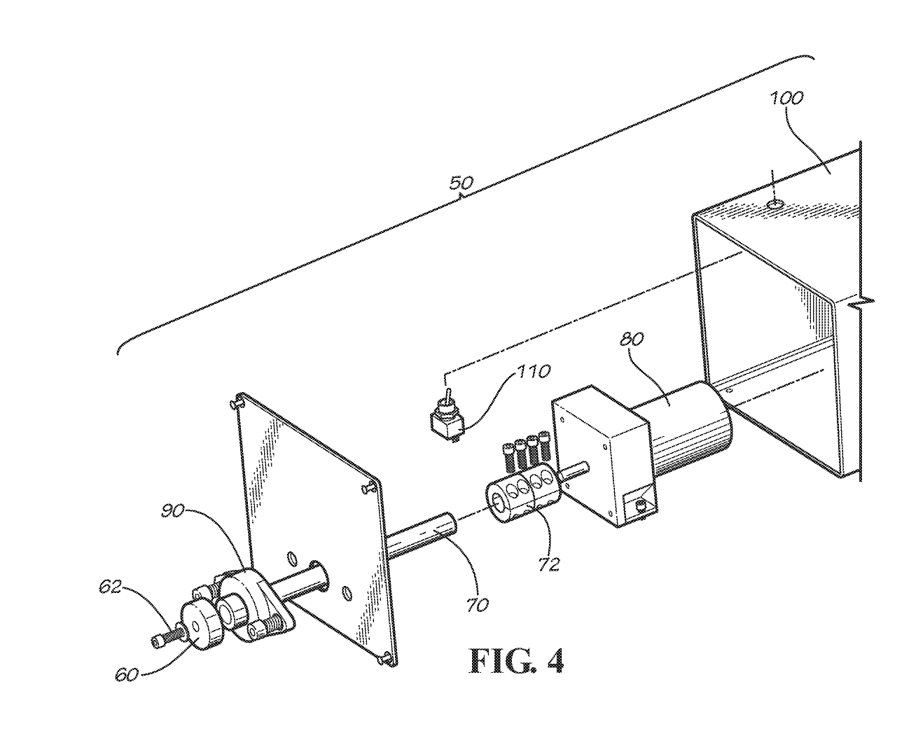

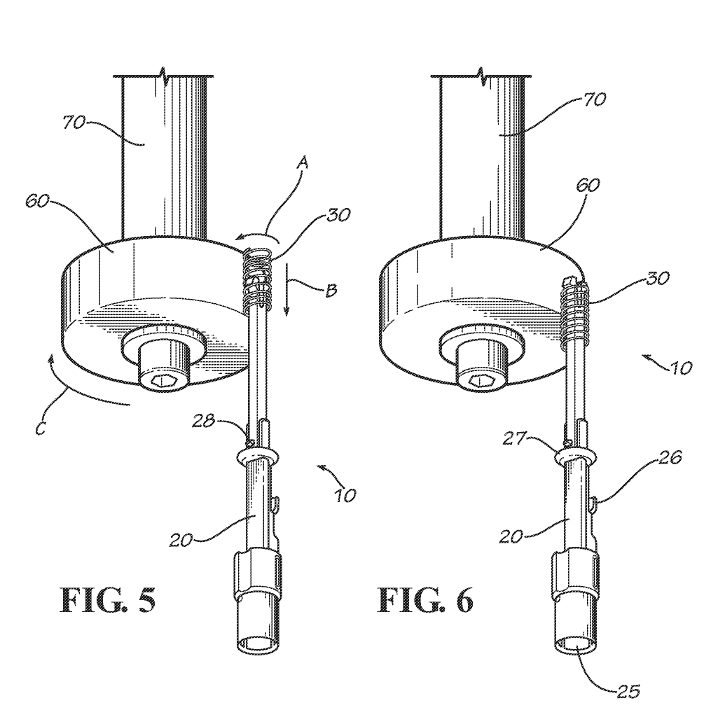

FIGS. 3 and 4 show an assembly system or spring winding device 50 according to an example form of the invention. The purpose of the spring winding device 50 is to assemble a spring onto a post or shaft having one-way snaps at the end, without damaging the spring or the shaft that the spring is installed onto. FIGS. 1 and 5 show the spring partially installed, and FIGS. 2 and 6 show the spring fully installed onto the post of the device in greater detail. FIG. 3 shows the assembly system 50 according to an example form, and FIG. 4 shows an assembly or exploded view of the system 50.

The assembly system 50 comprises a frictional disk 60 (alternately referred to as a drum or wheel) attached to a shaft 70 that rotates at a constant or variable speed, driven by a drive motor 80. The disk 60 is formed of a rubber, elastomer, soft plastic or polymer, and/or other material(s) having a durometer or hardness that is soft and flexible enough so that the spring does not slip on the disk, and hard and stiff enough to hold its shape during use and not wear prematurely. A material hardness of the disk 60 of between about 35A-95A Shore A durometer, optionally in particular between about 35A-65A Shore A durometer, and more particularly about 40A-50A Shore A durometer, with 40A and 50A Shore A durometer as particular examples, may be utilized to provide acceptable results in example applications. The disk 60 comprises a cylindrical body having an outer radius defining a circular circumferential contact face and having a disk thickness between first and second edges. The disk 60 is preferably a replaceable component that is detachably coupled to the motor by one or more screws 62 installed through a central bore through the disk, or other coupling elements. Transmission components such as one or more drive shafts 70, bushings 72, bearings 90, gearing, clutch mechanisms, couplings and the like are optionally installed between the disk 60 and the drive motor 80. The motor and drive transmission components are optionally at least partially contained within a housing 100. Power is provided to the motor via a cord or other coupling for connection to an external power source, or from a battery or other onboard power supply. A switch or other controller 110 is optionally provided for selectively actuating and/or controlling the speed of the system.

In an example method of operation, the disk 60 is brought into engagement with the spring 30 as shown in FIG. 5. Friction causes the spring 30 to thread itself onto the lancet holder 20, with both a twisting or rotational direction indicated by arrow A, and a translational direction indicated by arrow B, under the influence of the rotation of the disk 60 (indication by arrow C). The user places the lead-in end or first few coils of the spring onto the lancet holder and the spring winder finishes the assembly sequence of winding the spring in an automated fashion. Alternatively, the initial application of the spring onto the lancet holder is automated. Application of the spring winder to the spring can be manually carried out or alternatively is automated.

During the assembly process, the retainers 24 of the lancet carrier component 20 are preferably oriented generally tangentially to the surface of the disc 60 and the lengthwise axis of the shaft 22 is positioned generally parallel to the outer radial surface of the disc and spaced slightly away from the disc. In this manner, the disc 60 contacts the outer surface of the spring 30 and twists the spring while the shaft 22 remains stationary, with the stationary retainers 24 extending between the twisting helical coils of the rotating spring functioning as threads to wind the spring onto the carrier component 20, into the completed configuration shown in FIG. 6. The invention includes installation of one or more springs onto a carrier component. For example, a first spring comprising a drive spring for advancing a lancet can be installed onto a carrier, substantially in the manner described, and a second spring can be installed onto the carrier, either in series or in parallel with the first spring, in like manner.

The present invention includes the assembly system and method described herein, as well as various forms of lancing devices and other products and subassemblies assembled using the assembly system and method. A lancing device according to the present invention includes at least one spring, the spring being wound onto a lancet carrier or other component part of the lancet drive system or lancing device using the system and method of the invention as described.

While the invention has been described with reference to preferred and example embodiments, it will be understood by those skilled in the art that a variety of modifications, additions and deletions are within the scope of the invention, as defined by the following claims. For example, while the invention has been described above according to an example embodiment wherein the spring is wound onto the lancet carrier by contact of the rotating disk with the spring, in alternate forms of the invention the lancet carrier is rotated into the spring by contacting the rotating disk with the lancet carrier. In still further embodiments of the invention, the lancet carrier and the spring are each rotated, either in different directions or in the same direction at different speeds, by separate rotating disks and/or by different contact face portions of the same rotating disk.

What is claimed is:

1. A system for assembly of a lancing device, the lancing device comprising a carrier component and at least one spring to be mounted to the carrier component, the system comprising a disk for engaging at least a portion of the spring, and a drive mechanism for rotationally driving the disk, wherein the disk has a hardness selected to grip the spring and mount it onto the lancet carrier.

2. The system of claim 1, wherein the disk at least partially comprises rubber.

3. The system of claim 1, wherein the drive mechanism comprises an electric motor.

4. The system of claim 1, wherein the drive mechanism is at least partially contained in a housing.

5. The system of claim 1, wherein the disk comprises a contact surface having a Shore A durometer of between about 35-95.

6. The system of claim 1, wherein the disk comprises a contact surface having a Shore A durometer of between about 35-65.

7. The system of claim 1, wherein the disk comprises a contact surface having a Shore A durometer of between about 40-50.

8. The system of claim 1, wherein the disk comprises a contact surface having a Shore A durometer of between about 40.

9. The system of claim 1, wherein the disk comprises a contact surface having a Shore A durometer of between about 50.

10. A system for assembly of a device having a carrier component comprising a shaft and a retainer portion at an end of the shaft, and further comprising a spring mounted onto the shaft of the carrier component, the system comprising:
a disk having an outer radial contact face comprising a material having a Shore A durometer of between about 35-95; and
a motor coupled to the disk to rotationally drive the disk;
wherein application of the spring against the contact face of the disk spins the spring over the retainer portion of the carrier component and onto its shaft.

11. The system of claim 10, wherein the outer radial contact face has a Shore A durometer of between about 35-65.

12. The system of claim 10, wherein the outer radial contact face has a Shore A durometer of between about 40-50.

13. A method of assembly of a lancing device, the assembly method comprising applying a spring into partial assembly with a carrier component of a lancing device, engaging the spring with a rotating disk portion of a lancing device assembly system, and further assembling the spring onto the carrier component by operation of the rotating disk portion on the spring.

14. The assembly method of claim 13, wherein the step of applying a spring into partial assembly with a lancet drive component of a lancing device comprises manually installing a lead-in portion of the spring onto the lancet drive component.

15. A lancing device comprising a carrier component and at least one spring, wherein the lancing device is assembled by installing the at least one spring onto the carrier component by winding the spring onto the carrier component using a rotating disk that is applied against at least a portion of the spring and/or against at least a portion of the carrier component.

16. The lancing device of claim 15, wherein the lancet carrier comprises a forked end having first and second prongs, each of the first and second prongs having a barbed retainer thereon for retaining the spring.

17. The lancing device of claim 16, wherein the lancet carrier further comprises a cylindrical receiver or resilient collar opposite the forked end for releasably engaging a disposable lancet.

18. The lancing device of claim 17, wherein the lancet carrier further comprises a cantilevered arm extending therefrom at a medial portion between the cylindrical receiver and the forked end.

19. The lancing device of claim 17, wherein the lancet carrier further comprises a circumferential flange projecting outwardly from a medial shaft portion between the cylindrical receiver and the forked end.

20. The lancing device of claim 15, comprising a drive spring and a return spring, the drive spring being installed around a first shaft portion of the lancet carrier and the return spring being installed around a second shaft portion of the lancet carrier.
